(19) United States
(12) Patent Application Publication
(10) Pub. No.: US 2007/0206723 A1
(43) Pub. Date: Sep. 6, 2007

(54) Mammographic system and breast compression plate for use in mammographic system

(75) Inventors: Naoyuki Okada, Minami-ashigara-shi (JP); Tomonari Sendai, Minami-ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/712,503

(22) Filed: Mar. 1, 2007

(30) Foreign Application Priority Data
Mar. 1, 2006 (JP) 2006-055585

(51) Int. Cl. A61B 6/04

(52) U.S. Cl. 378/37

(57) ABSTRACT

A mammographic system for capturing a radiation image of the breast of an examinee includes an image capturing base, having a radiation image information acquisition unit for acquiring information of the radiation image, and a breast compression plate for compressing the breast against the image capturing base. The breast compression plate has a fixed base for compressing and securing the breast, the fixed base includes a surface to be held against a chest wall of the examinee, and a movable flap connected to the fixed base, which is displaceable in order to release the breast.

### BACKGROUND OF THE INVENTION

1. Field of the Invention

The present invention relates to a mammographic system for capturing a radiation image of the breast of an examinee, together with a breast compression plate for use in such a mammographic system.

2. Description of the Related Art

Generally, mammographic systems employ a breast compression plate for compressing the breast of an examinee against an image capturing base. The breast compression plate is disposed between a radiation source that emits radiation and the image capturing base, which incorporates a detector for detecting the radiation emitted from the radiation source. The breast compression plate compresses the breast, which is to be imaged, into a flatter shape having a substantially uniform thickness, so that a better radiation image of the breast can be obtained.

Japanese Laid-Open Patent Publication No. 4-64346 discloses a breast compression mechanism for lowering a breast compression plate mounted on a vertically movable support member to compress the breast. Japanese Laid-Open Utility Model Publication No. 6-38916 reveals a breast compression plate whose angle can be adjusted by an angle adjuster, which is disposed between the breast compression plate and a support bar supporting the breast compression plate.

For securing the breast in position with a breast compression plate, it is the customary practice for the operator to hold the breast by hand, and then to compress and secure the breast with the breast compression plate while keeping the breast held by hand. After having secured the breast in position, the operator's hand, which has been compressed or sandwiched together with the breast by the breast compression plate, has to be removed from the breast compression plate.

The breast compression plate disclosed in Japanese Laid-Open Patent Publication No. 4-64346 compresses and secures the breast parallel to the image capturing base. After the breast is compressed and secured in position by the breast compression plate, it is hard for the operator to remove his or her hand, and both the operator and the examinee are liable to feel pain when the hand is removed.

The breast compression plate disclosed in Japanese Laid-Open Utility Model Publication No. 6-38916 can be angularly adjusted to the examinee's body type. After adjusting the angle of the breast compression plate to fit the examinee's body type, the angle adjuster is fastened, and then the breast is compressed by the breast compression plate. Therefore, the operator still finds it hard to remove his or her hand from the breast compression plate.

U.S. Pat. No. 5,506,877 shows a breast compression paddle whose angle can be adjusted dependent on the gradient of the breast toward the nipple. The disclosed breast compression paddle is effective to compress small-sized breasts. The angle adjustability of the breast compression paddle provides a function to secure the breast uniformly in position. However, there is nothing taken into account to make it easy for the operator to remove his or her hand from the breast compression paddle. Therefore, the operator finds it hard to remove his or her hand from the breast compression paddle after the angularly adjusted breast compression paddle has been secured in place. Furthermore, the breast compression paddle is formed of a single plate, wherein the entire surface thereof is used to compress and secure the breast. Consequently, if the breast compression paddle is angularly loosened to allow the operator to remove his or her hand more easily, then the breast that has been secured in position tends to be displaced.

### SUMMARY OF THE INVENTION

It is a general object of the present invention to provide a breast compression plate, which allows the operator's hand to be removed easily while a breast is being secured in a desired position on the breast compression plate and while the breast is compressed by the breast compression plate. The present invention also has the object of providing a mammographic system, which incorporates such a breast compression plate.

Another object of the present invention is to provide a breast compression plate that reduces the burden on the operator who works with the breast compression plate, as well as the burden on the examinee whose breast is being compressed by the breast compression plate, together with a mammographic system incorporating such a breast compression plate.

Still another object of the present invention is to provide a breast compression plate which is capable of positioning a breast of an examinee highly accurately, together with a mammographic system incorporating such a breast compression plate.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a perspective view of a mammographic system according to a first embodiment of the present invention;

FIG. 2 is a fragmentary vertical elevational view, partly in cross section, showing internal structural details of an image capturing base of the mammographic system shown in FIG. 1;

FIG. 3 is a perspective view of a breast compression plate of the mammographic system shown in FIG. 1;

FIG. 4 is a plan view of the breast compression plate shown in FIG. 3;

FIG. 5 is a perspective view of the breast compression plate shown in FIG. 3, with movable flaps thereof displaced;

FIG. 6 is a side elevational view of the breast compression plate shown in FIG. 3, with the movable flaps thereof displaced;

FIG. 7 is a cross-sectional view taken along line VII-VII of FIG. 4;

FIG. 8 is a plan view of a breast compression plate according to a second embodiment of the present invention;

FIG. 9 is a perspective view of the breast compression plate shown in FIG. 8, with movable flaps thereof displaced;

FIG. 10 is a side elevational view of the breast compression plate shown in FIG. 8, with the movable flaps thereof displaced;

FIG. 11 is a plan view of a breast compression plate according to a third embodiment of the present invention;

FIG. 12 is a perspective view of the breast compression plate shown in FIG. 11, with movable flaps thereof displaced;

FIG. 13 is a side elevational view of the breast compression plate shown in FIG. 11, with the movable flaps thereof displaced;

FIG. 14 is a plan view of a breast compression plate according to a fourth embodiment of the present invention;

FIG. 15 is a perspective view of the breast compression plate shown in FIG. 14, with movable flaps thereof displaced;

FIG. 16 is a plan view of a breast compression plate according to a fifth embodiment of the present invention;

FIG. 17 is a perspective view of the breast compression plate shown in FIG. 16, with movable flaps thereof displaced;

FIG. 18 is a plan view of a breast compression plate according to a sixth embodiment of the present invention;

FIG. 19 is a perspective view of the breast compression plate shown in FIG. 18, with movable flaps thereof displaced; and

FIG. 20 is a perspective view of a modified breast compression plate according to the present invention.

### DESCRIPTION OF THE PREFERRED EMBODIMENTS

Mammographic systems according to embodiments of the present invention, along with breast compression plates incorporated therein, shall be described in detail below with reference to the accompanying drawings.

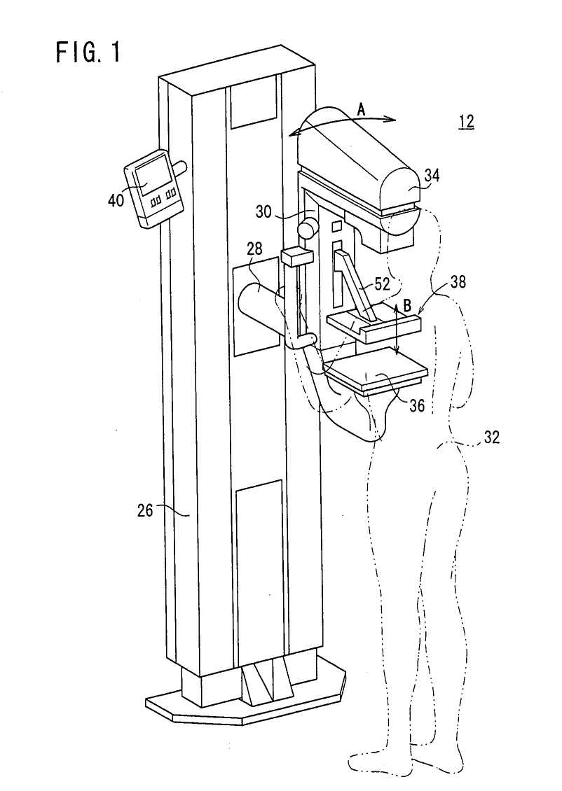

A mammographic system 12, shown in FIG. 1, according to a first embodiment of the present invention serves to capture radiation image information of a breast, which is the subject of examination.

As shown in FIG. 1, the mammographic system 12 includes an upstanding base 26, a vertical arm 30 fixed to a horizontal swing shaft 28 disposed substantially centrally on the base 26, a radiation source housing unit 34 fixed to an upper end of the arm 30, an image capturing base 36 housing therein a solid-state detector for detecting radiation that has passed through the examinee 32 and which is fixed to a lower end of the arm 30, and a breast compression plate 38 for compressing and holding the breast of the examinee 32 against the image capturing base 36. The radiation source housing unit 34 stores a radiation source for applying radiation to the breast of the examinee 32. The image capturing base 36 houses therein a solid-state detector for detecting the radiation that has passed through the examinee 32 in order to acquire radiation image information of the breast.

When the arm 30, to which the radiation source housing unit 34, the image capturing base 36, and the breast compression plate 38 are secured, is angularly moved about the swing shaft 28 in a direction indicated by the arrow A, an image capturing direction with respect to the breast of the examinee 32 can be adjusted.

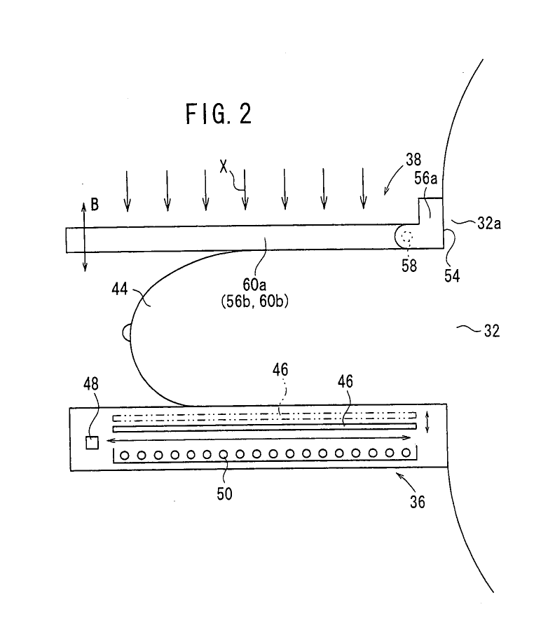

FIG. 2 shows internal structural details of the image capturing base 36 of the mammographic system 12. In FIG. 2, the breast 44 of the examinee 32 that is to be imaged is shown as being placed between the image capturing base 36 and the breast compression plate 38.

The image capturing base 36 houses therein a solid-state detector 46 (radiation image information acquisition unit) for storing radiation image information based on radiation X emitted from the radiation source of the radiation source housing unit 34 and outputting the stored radiation image information as an electric signal, a reading light source 48 for applying a reading light to the solid-state detector 46 in order to read the radiation image stored by the solid-state detector 46, and an erasing light source 50 for applying erasing light to the solid-state detector 46 to remove unwanted electric charges accumulated within the solid-state detector 46.

The solid-state detector 46 comprises a direct-conversion, light-reading radiation solid-state detector. The solid-state detector 46 stores radiation image information represented by the radiation X that has passed through the breast 44, which is compressed into a flat shape and held by the breast compression plate 38, as an electrostatic latent image, and generates a current that depends on the electrostatic latent image when the solid-state detector 46 is scanned by the reading light applied from the reading light source 48. For recording radiation image information, the solid-state detector 46 is moved to a position near the breast 44. When unwanted electric charges are to be erased from the solid-state detector 46, the solid-state detector 46 is moved to a position near the erasing light source 50.

More specifically, the solid-state detector 46 comprises a laminated assembly made up of a first electrically conductive layer disposed on a glass substrate for passing the radiation X therethrough, a recording photoconductive layer for generating electric charges upon exposure to the radiation X, a charge transport layer, which acts substantially as an electric insulator with respect to latent image polarity electric charges developed in the first electrically conductive layer, and which acts substantially as an electric conductor with respect to transport polarity charges that are of a polarity opposite to the latent image polarity electric charges, a reading photoconductive layer for generating electric charges and becoming electrically conductive upon exposure to the reading light, and a second electrically conductive layer that is permeable to the radiation X. An electric energy storage region is provided in the interface between the recording photoconductive layer and the charge transport layer.

The first electrically conductive layer and the second electrically conductive layer each provides an electrode. The electrode provided by the first electrically conductive layer comprises a two-dimensional flat electrode, whereas the electrode provided by the second electrically conductive layer comprises a plurality of linear electrodes spaced at a predetermined pixel pitch, for detecting radiation image information to be recorded as an image signal. The linear electrodes are arranged in an array along a main scanning direction, and extend in an auxiliary scanning direction perpendicular to the main scanning direction (for details, see Japanese Laid-Open Patent Publication No. 2004-154409, for example).

The reading light source 48 includes, for example, a line light source comprising a linear array of LED chips, and an optical system for applying a line of reading light emitted from the line light source to the solid-state detector 46. The linear array of LED chips extends perpendicularly to the direction in which the linear electrodes of the second electrically conductive layer of the solid-state detector 46 extend. The line light source moves along the direction in which the linear electrodes extend, so as to expose and scan the entire surface of the solid-state detector 46.

The erasing light source 50 should preferably comprise a light source that can emit and quench light in a short period of time and which has very short persistence. For example, the erasing light source 50 may comprise a plurality of external-electrode rare-gas fluorescent lamps extending along the direction of the array of LED chips of the reading light source 48, and arranged in an array perpendicular to the direction of the array of LED chips of the reading light source 48.

The breast compression plate 38 is connected to the arm 30 by a support arm 52, and is disposed between the radiation source housing unit 34 and the image capturing base 36. The breast compression plate 38 is vertically displaceable along the arm 30, in the directions indicated by the arrow B, by the support arm 52 so as to be movable or displaceable in the directions indicated by the arrow B.

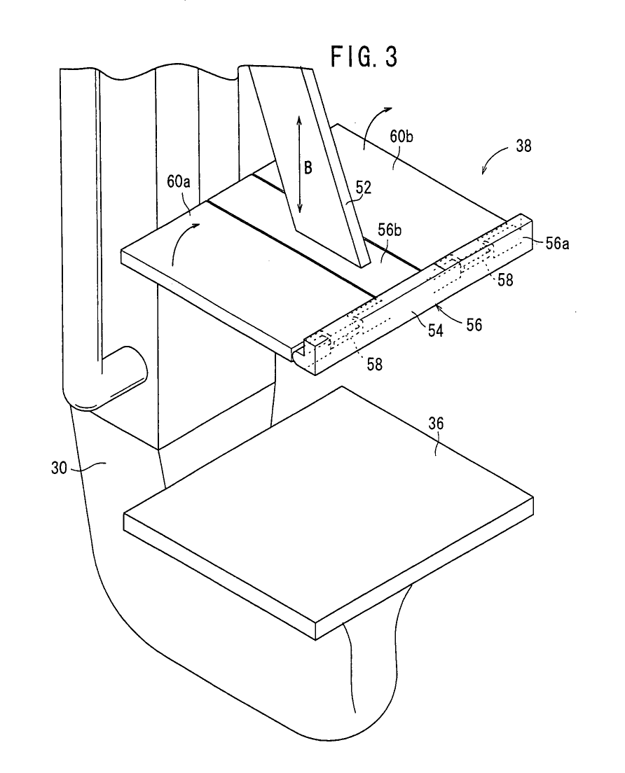

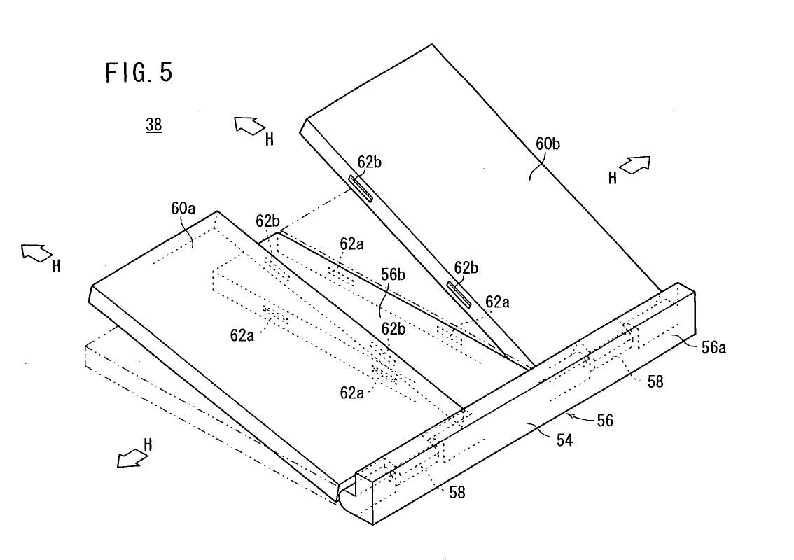

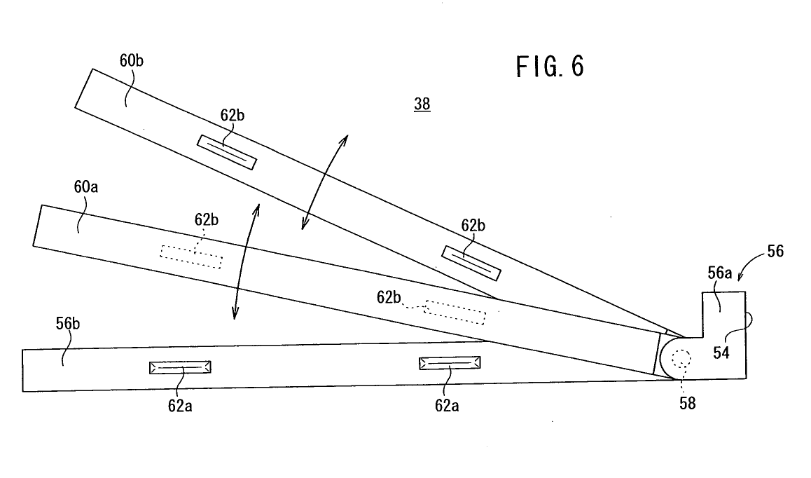

The breast compression plate 38 shall be described in detail below with reference to FIGS. 2 through 7. As shown in FIGS. 2 and 3, the breast compression plate 38 according to the present embodiment comprises a fixed base 56, which is substantially T-shaped as viewed in plan, and has an abutment surface 54 that is to be held against a chest wall 32a of the examinee 32. The fixed base 56 is supported by the support arm 52, and a pair of movable flaps 60a, 60b are movably connected to the fixed base 56. The fixed base 56 includes a central main compression foot 56b secured to the lower end of the support arm 52, and a base bar 56a connected to the front end of the main compression foot 56b and having the abutment surface 54. The movable flaps 60a, 60b are disposed one on each side of the main compression foot 56b, and are pivotally mounted on the base bar 56a by respective hinges (pivot mechanism) 58 on the base bar 56a. the movable flaps 60a, 60b are angularly movable about the hinges 58 away from the image capturing base 36 (see FIGS. 5 and 6).

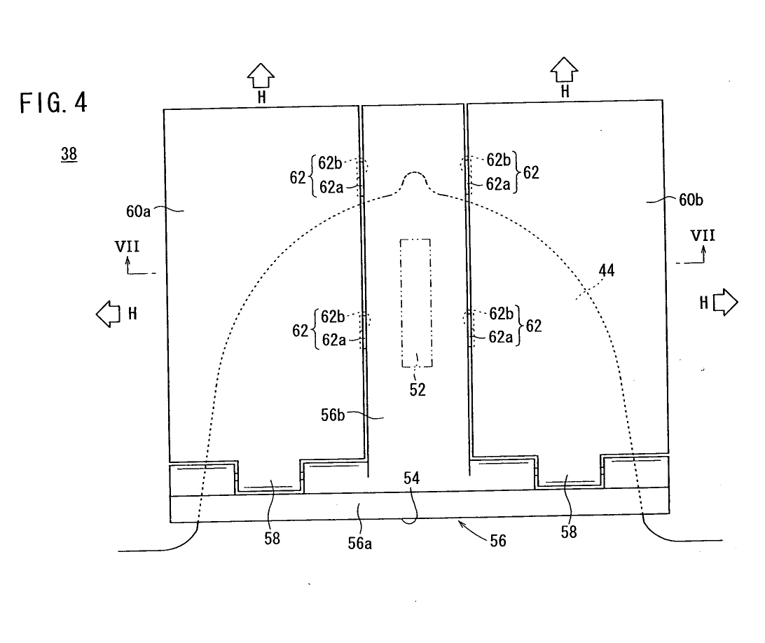

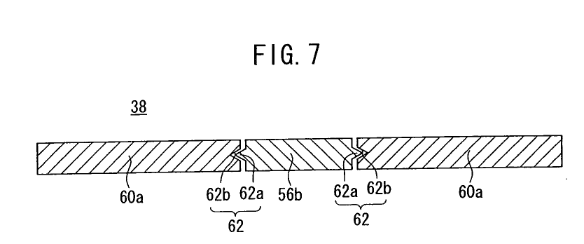

Two lock mechanisms 62 are disposed between the main compression foot 56b of the fixed base 56 and the movable flaps 60a, 60b (see FIG. 4). The lock mechanisms 62 function to lock, i.e., secure, the movable flaps 60a, 60b to the fixed base 56, and more specifically the main compression foot 56b thereof, when the breast 44 is compressed and secured in position while a radiation image of the breast 44 is captured. The lock mechanisms 62 comprise respective pairs of ridges (second engaging members) 62a projecting respectively on opposite longitudinal side surfaces of the main compression foot 56b, each in the form of a horizontal triangular prism, and respective pairs of recesses (first engaging members) 62b defined respectively on side surfaces of the movable flaps 60a, 60b which face the main compression foot 56b. The ridges 62a are engageable within the corresponding recesses 62b (see also FIG. 7). When the movable flaps 60a, 60b are angularly moved about the hinges 58, the movable flaps 60a, 60b can easily be placed into and out of locked engagement with the main compression foot 56b, i.e., the fixed base 56.

To the base 26, there is connected a display control panel 40 for displaying image capturing information including an image capturing region, an image capturing direction, etc., of the examinee 32, and ID information of the examinee 32, etc., which have been detected by the mammographic system 12, and for setting these items of information as necessary.

The mammographic system 12 according to the present embodiment is basically constructed as described above. Operations of the mammographic system 12 shall be described below.

Using a console (not shown) and/or an ID card, etc., the operator sets ID information of the examinee 32, and image capturing information, such as various image capturing process details, etc. The ID information represents the name, age, sex, etc., of the examinee 32, and can be acquired from an ID card possessed by the examinee 32. The image capturing process details include an image capturing region, an image capturing direction, etc., which are specified by the doctor in charge of the examinee 32, and are entered by the operator, for example, using the console. These entered items of information can be displayed for confirmation on the display control panel 40 of the mammographic system 12.

Then, the operator places the mammographic system 12 in a certain state in accordance with the specified image capturing direction. For example, the breast 44 may be imaged as a cranio-caudal view (CC) taken from above, a medio-lateral view (ML) taken outwardly from the center of the chest, or a medio-lateral oblique view (MLO) taken from an oblique view. Depending on information of the selected one of these image capturing directions, the operator turns the arm 30 about the swing shaft 28.

The operator then places the breast 44 into a specified image capturing state with respect to the mammographic system 12. For example, if the breast 44 of the examinee 32 is to be imaged as a cranio-caudal view (CC), the operator places the breast 44 on the image capturing base 36, and thereafter lowers the breast compression plate 38 to hold the breast 44 between the image capturing base 36 and the breast compression plate 38, as shown in FIG. 2.

Specifically, the operator holds the breast 44 in a desired position on the image capturing base 36 by hand, and then lowers the breast compression plate 38 while holding the breast 44, thereby compressing and securing the breast 44 with the operator's hand remaining pinched in between. The operator then must remove his or her hand from the breast compression plate 38.

When the breast compression plate 38 is lowered to compress the breast 44, while the breast 44 is being held by hand, a proximal portion of the breast 44 which is closer to the chest wall 32a is firmly held in its transverse direction by the base bar 56a of the fixed base 56, whereas a transversely central portion of the breast 44, which ranges from the proximal to distal ends thereof, is firmly held by the main compression foot 56b of the fixed base 56.

At this time, the movable flaps 60a, 60b are unlocked from the main compression foot 56b and are angularly displaced about the hinges 58 away from the image capturing base 36. Therefore, the hand of the operator, which is holding the breast 44, is not excessively compressed by the movable flaps 60a, 60b. The operator can thus easily remove his or her hand in any one of the directions indicated by the arrows H shown in FIGS. 4 and 5. At the same time, as described above, the proximal portion of the breast 44 and the transversely central portion of the breast 44, which ranges from the proximal to distal ends thereof, remain firmly held by the fixed base 56. Consequently, the breast 44 is firmly held in position and prevented from unwanted displacement when the operator pulls his or her hand away from the breast compression plate 38.

Then, the movable flaps 60a, 60b are lowered to compress the breast 44 uniformly until they are locked to the main compression foot 56b by the lock mechanisms 62. The breast 44 is now reliably secured in position between the breast compression plate 38 and the image capturing base 36.

The breast compression plate 38 according to the present embodiment prevents the operator's hand from becoming pinched excessively between the breast compression plate 38 and the image capturing base 36, and hence prevents the breast 44 from being excessively compressed by the operator's hand. The operator can thus remove his or her hand from the breast compression plate 38 within a short period of time. The burden on the operator who works with the breast compression plate 38 is also greatly reduced, and any pain felt by the operator when his or her hand is placed between the breast compression plate 38 and the image capturing base 36 is also significantly reduced. The burden imposed on the examinee 32, and pain felt when the examinee's breast 44 is compressed, also are greatly reduced.

If the breast 44 is of a relatively small size, then the operator may need to pull the breast 44 strongly when the breast 44 is to be secured in a desired position on the image capturing base 36. In this case, when the operator pulls his or her hand from the breast compression plate 38, the breast 44 is highly likely to be displaced in position. However, the breast compression plate 38 according to the present embodiment allows the operator to remove his or her hand from the breast compression plate 38 while the proximal portion of the breast 44 is firmly held by the fixed base 56, and while the transversely central portion of the breast 44, which ranges from the proximal to distal ends thereof, is firmly held by the fixed base 56. Accordingly, the breast compression plate 38 is also effective to compress a breast 44 of a relatively small size.

After the image capturing region (attitude) of the examinee 32 has been determined, the radiation source within the radiation source housing unit 34 is energized in order to capture radiation image information.

Specifically, the radiation X emitted from the radiation source passes through the breast 44 that is held between the breast compression plate 38 and the image capturing base 36, and is applied to the solid-state detector 46 housed within the image capturing base 36. Before a radiation image is captured, the entire surface of the solid-state detector 46 is irradiated with erasing light from the erasing light source 50 in order to remove unwanted electric charges from the solid-state detector 46. Thereafter, the solid-state detector 46 is placed in the two-dot-and-dash-line position shown in FIG. 2. After the radiation X has passed through the breast 44, the radiation X carries radiation image information of the breast 44. When the radiation X, which carries the radiation image information of the breast 44, is applied to the solid-state detector 46 while a high voltage is being applied between the first electrically conductive layer and the second electrically conductive layer, pairs of positive and negative electric charges are generated within the recording photoconductive layer of the solid-state detector 46, and the negative electric charges are stored in the electric energy storage region that is provided in the interface between the recording photoconductive layer and the charge transport layer. In this manner, the radiation image information is captured.

Then, the reading light source 48 moves in a direction indicated by the arrow along the solid-state detector 46, while applying the reading light to the solid-state detector 46. The radiation image information is processed by a CAD system or the like (not shown) to automatically detect an affected region, if any, in the breast 44 and mark the affected region in the radiation image information. The doctor then diagnoses the radiation image information.

Thereafter, the solid-state detector 46 is irradiated with erasing light emitted from the erasing light source 50 in order to remove unwanted electric charges that have accumulated within the solid-state detector 46, for thereby preparing the solid-state detector 46 for a next radiation image information capturing cycle.

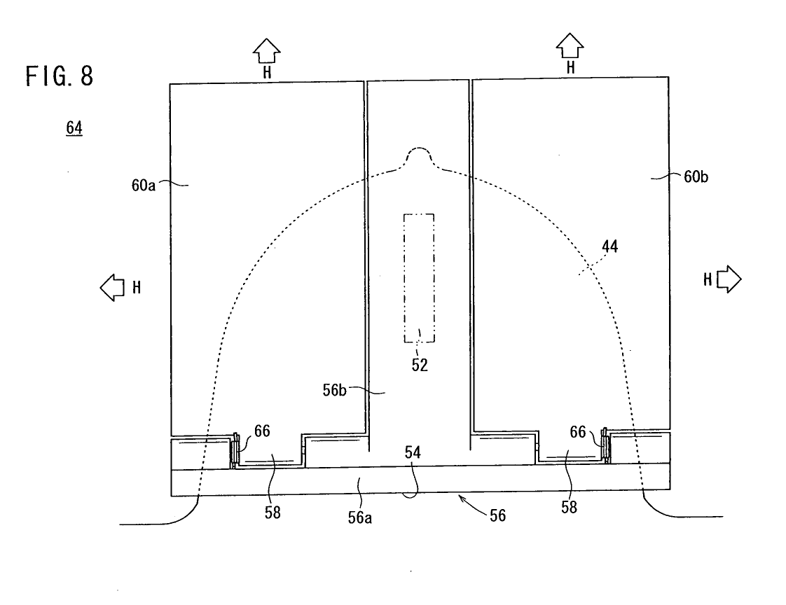

A breast compression plate according to a second embodiment of the present invention shall be described below with reference to FIGS. 8 through 10. Reference characters in FIGS. 8 through 10, which are identical to those shown in FIGS. 1 through 7, are intended to denote identical or similar structural details having identical or similar functions and advantages. Such identical reference characters, denoting identical or similar structural details having identical or similar functions and advantages, also are applicable to the other embodiments.

A breast compression plate 64 according to the second embodiment differs from the breast compression plate 38 according to the first embodiment in that helical torsion springs 66 (resilient members) are disposed respectively near the hinges 58. Further, the breast compression plate 64 is free of the lock mechanisms 62.

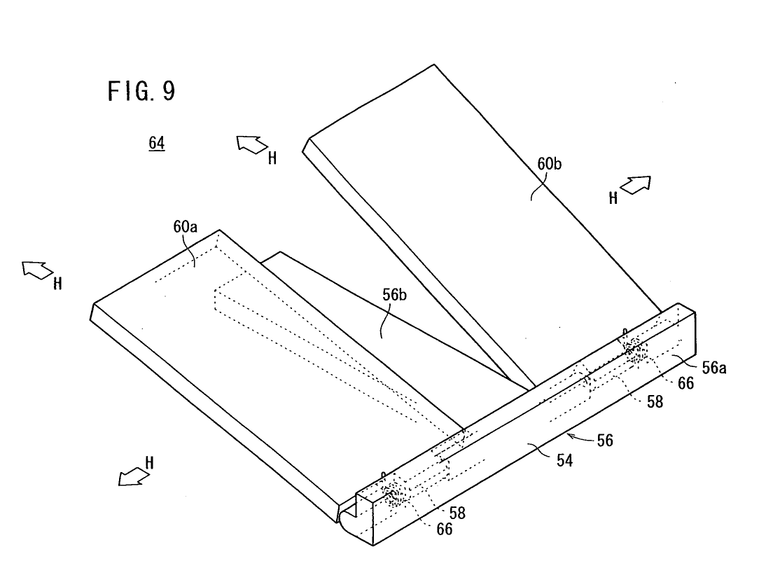

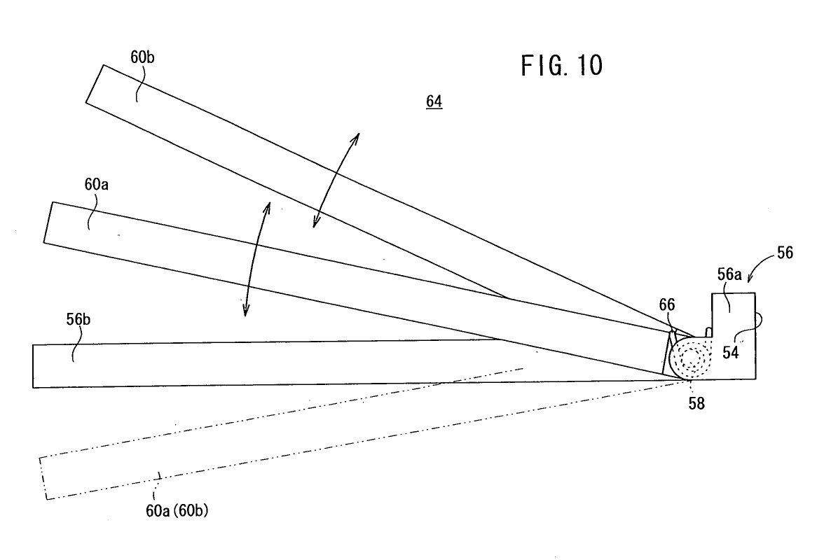

The helical torsion springs 66 act between the movable flaps 60a, 60b and the base bar 56a so as to normally bias the movable flaps 60a, 60b to turn toward the image capturing base 36. When the breast 44 that is held by the hand of the operator is compressed by the breast compression plate 64, the proximal portion of the breast 44 and the transversely central portion of the breast 44, which ranges from the proximal to distal ends thereof, are firmly secured in position by the fixed base 56. At this time, the movable flaps 60a, 60b are angularly displaced away from the image capturing base 36 against the bias of the helical torsion springs 66, while lightly compressing the hand of the operator (see FIGS. 9 and 10).

Therefore, the hand of the operator and the breast 44 are not excessively compressed. The operator can easily remove his or her hand in any one of the directions indicated by the arrows H shown in FIGS. 8 and 9. After the hand of the operator is removed, the movable flaps 60a, 60b uniformly compress the breast 44 under the bias of the helical torsion springs 66. The breast 44 is thus firmly secured in a desired position between the breast compression plate 64 and the image capturing base 36.

In the mammographic system 12 incorporating the breast compression plate 64 according to the second embodiment, the operator's hand is prevented from becoming pinched excessively between the breast compression plate 64 and the image capturing base 36, and hence the breast 44 is prevented from being excessively compressed by the operator's hand. The operator can remove his or her hand from the breast compression plate 64 in a short period of time. The burden on the operator who works with the breast compression plate 64 is greatly reduced, and any pain felt by the operator when his or her hand is placed between the breast compression plate 64 and the image capturing base 36 is also significantly reduced. The burden imposed on the examinee 32, and pain felt when the examinee's breast 44 is compressed, also are greatly reduced. The breast compression plate 64 according to the second embodiment may include the lock mechanisms 62.

In the breast compression plate 64 according to the second embodiment, the movable flaps 60a, 60b may be angularly displaced downwardly from their position horizontally aligned with the main compression foot 56b toward the image capturing base 36, as indicated by the two-dot-and-dash lines shown in FIG. 10. The movable flaps 60a, 60b, which may thus be angularly displaced downwardly, are effective in more firmly securing the breast 44, which has a shape slanting downwardly from the proximal portion toward the nipple region thereof.

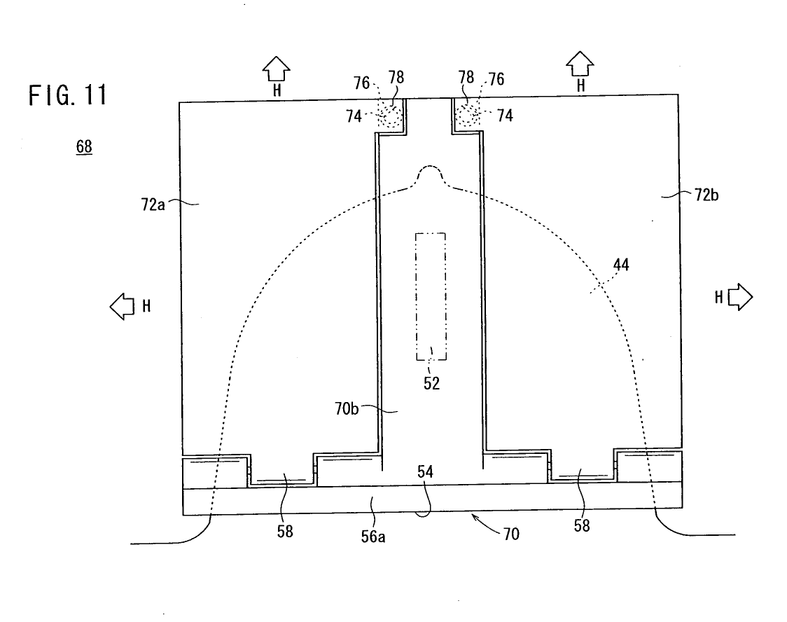

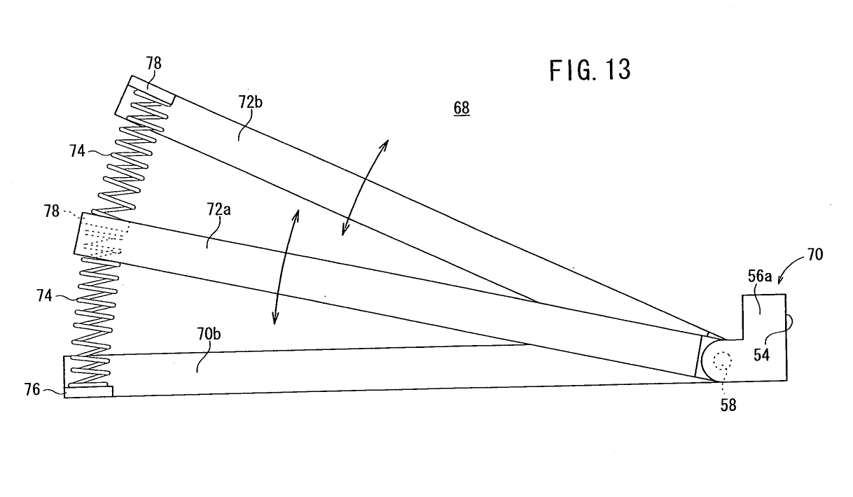

A breast compression plate according to a third embodiment of the present invention will be described below with reference to FIGS. 11 through 13.

The breast compression plate 68 according to the third embodiment differs from the breast compression plate 38 according to the first embodiment in that it has a fixed base 70 and movable flaps 72a, 72b, in place of the fixed base 56 and the movable flaps 60a, 60b, and also is free of the lock mechanisms 62.

The fixed base 70 has a main compression foot 70b including two laterally spaced mounts (second mounts) 76, which are spaced or offset from an upper surface of the main compression foot 70b by a predetermined distance toward the image capturing base 36, at an end of the main compression foot 70b remote from the base bar 56a, i.e., at an end of the main compression foot 70b closer to the nipple region of the breast 44 as it is compressed by the main compression foot 70b. Helical springs 74 (extensible and contractible members) have respective lower ends fixed to the respective mounts 76.

The movable flaps 72a, 72b have respective mounts (first mounts) 78 at corners thereof, which are remote from the fixed base 70 and face each other. When the movable flaps 72a, 72b are in their home position, i.e., the position where the movable flaps 72a, 72b are turned through an angle of about 0°, i.e., when the movable flaps 72a, 72b lie substantially flush with the main compression foot 70b, the mounts 78 are positioned over the respective mounts 76 of the main compression foot 70b in vertical alignment therewith (see FIG. 11).

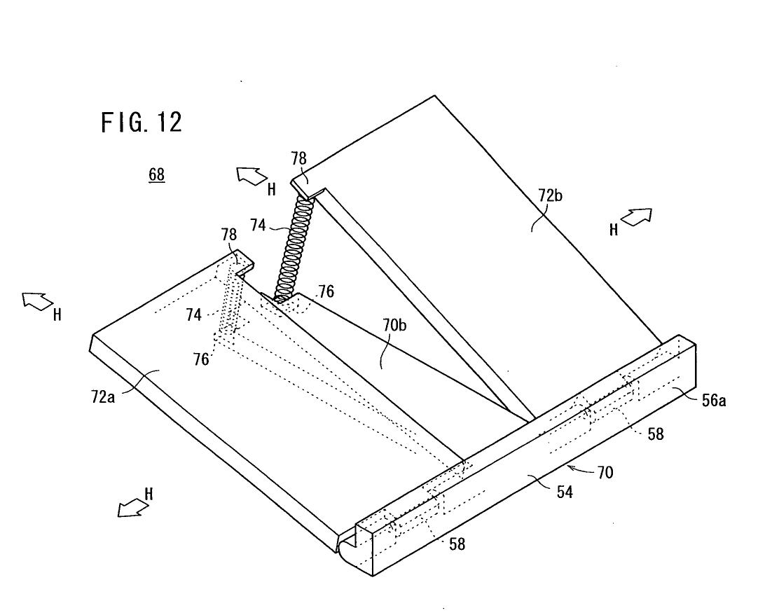

The helical springs 74 have respective upper ends fixed to the respective mounts 78. Therefore, the helical springs 74 act between the mounts 76 of the main compression foot 70b and the mounts 78 of the movable flaps 72a, 72b, for normally biasing the movable flaps 72a, 72b to turn toward the image capturing base 36. Consequently, when the breast 44 that is held by the hand of the operator is compressed by the breast compression plate 68, the proximal portion of the breast 44 and the transversely central portion of the breast 44, which ranges from the proximal to distal ends thereof, are firmly secured in position by the fixed base 70. At this time, the movable flaps 72a, 72b are angularly displaced away from the image capturing base 36 against the bias of the helical springs 74, while lightly compressing the hand of the operator. Therefore, the hand of the operator and the breast 44 are not excessively compressed. The operator can easily remove his or her hand in any one of the directions indicated by the arrows H shown in FIGS. 11 and 12. After the hand of the operator is removed, the movable flaps 72a, 72b uniformly compress the breast 44 under the bias of the helical springs 74. The breast 44 is thus firmly secured in a desired position between the breast compression plate 68 and the image capturing base 36.

In the mammographic system 12 incorporating the breast compression plate 68 according to the third embodiment, the operator's hand is prevented from becoming pinched excessively between the breast compression plate 64 and the image capturing base 36, and hence the breast 44 is prevented from being excessively compressed by the operator's hand. The operator can remove his or her hand from the breast compression plate 68 in a short period of time. The burden on the operator who works with the breast compression plate 68 is greatly reduced, and any pain felt by the operator when his or her hand is placed between the breast compression plate 68 and the image capturing base 36 is also significantly reduced. The burden imposed on the examinee 32, and pain felt when the examinee's breast 44 is compressed, also are greatly reduced. The breast compression plate 68 according to the third embodiment may include the lock mechanisms 62.

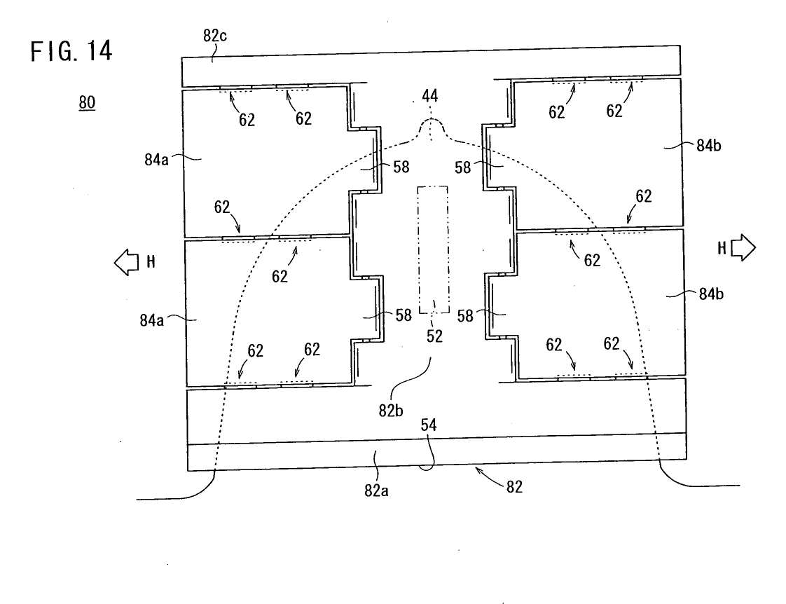

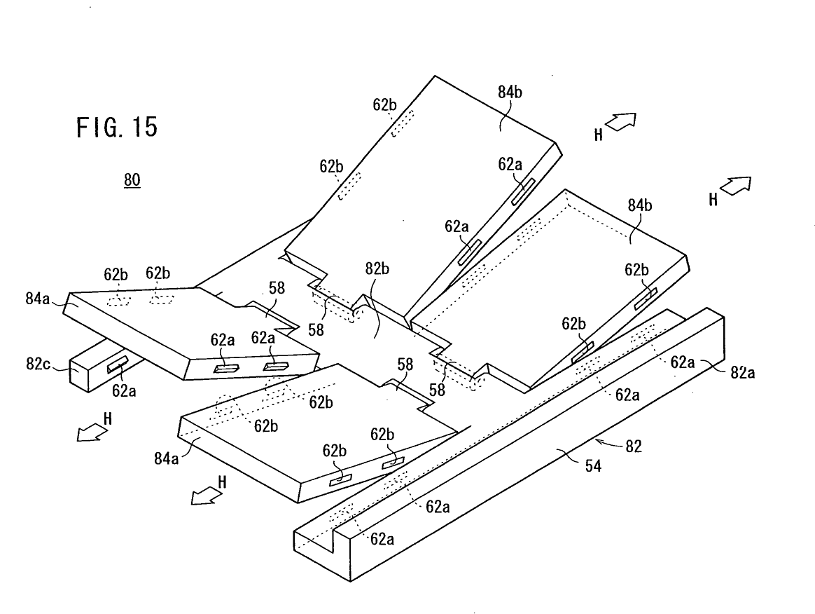

A breast compression plate according to a fourth embodiment of the present invention shall be described below with reference to FIGS. 14 and 15.

The breast compression plate 80 according to the fourth embodiment differs from the breast compression plate 38 according to the first embodiment in that it has a fixed base 82 and two pairs of movable flaps 84a, 84b, in place of the fixed base 56 and the movable flaps 60a, 60b.

As shown in FIGS. 14 and 15, the fixed base 82 is substantially H-shaped as viewed in plan and has a base bar 82a and a main compression foot 82b. The main compression foot 82b has a distal end bar 82c disposed remotely from and extending parallel to the base bar 82a. The main compression foot 82b has four hinges 58, two on each side. The movable flaps 84a, 84b are angularly movably mounted on the respective hinges 58 for enabling turning movement away from the image capturing base 36.

Specifically, the two movable flaps 84a are disposed on one side of the main compression foot 82b between the base bar 82a and the distal end bar 82c, whereas the two movable flaps 84a are disposed on the other side of the main compression foot 82b between the base bar 82a and the distal end bar 82c. Pairs of lock mechanisms 62 are disposed respectively between the base bar 82a and the two movable flaps 84a, 84b, between the distal end bar 82c and the two movable flaps 84a, 84b, between the two movable flaps 84a, and between the two movable flaps 84b.

When the breast 44 that is held in position by the hand of the operator is to be compressed by the breast compression plate 80, the lock mechanisms 62 are unlocked. The proximal portion of the breast 44 and the transversely central portion of the breast 44, which ranges from the proximal to distal ends thereof, are firmly secured in position by the fixed base 82. Since the movable flaps 84a, 84b are angularly moved, the hand of the operator and the breast 44 are not excessively compressed. The operator can thus easily remove his or her hand in any one of the directions indicated by the arrows H shown in FIGS. 14 and 15.

Then, when the movable flaps 84a, 84b are locked by the lock mechanisms 62 and while the breast 44 is uniformly compressed by the movable flaps 84a, 84b, the breast 44 is firmly secured in a desired position between the breast compression plate 80 and the image capturing base 36.

In the mammographic system 12 incorporating the breast compression plate 80 according to the fourth embodiment, the operator's hand is prevented from becoming pinched excessively between the breast compression plate 64 and the image capturing base 36, and hence the breast 44 is prevented from being excessively compressed by the operator's hand. The operator can remove his or her hand from the breast compression plate 80 in a short period of time. The burden on the operator who works with the breast compression plate 80 is greatly reduced, and any pain felt by the operator when his or her hand is placed between the breast compression plate 80 and the image capturing base 36 is also significantly reduced. The burden imposed on the examinee 32, and pain felt when the examinee's breast 44 is compressed, are also greatly reduced.

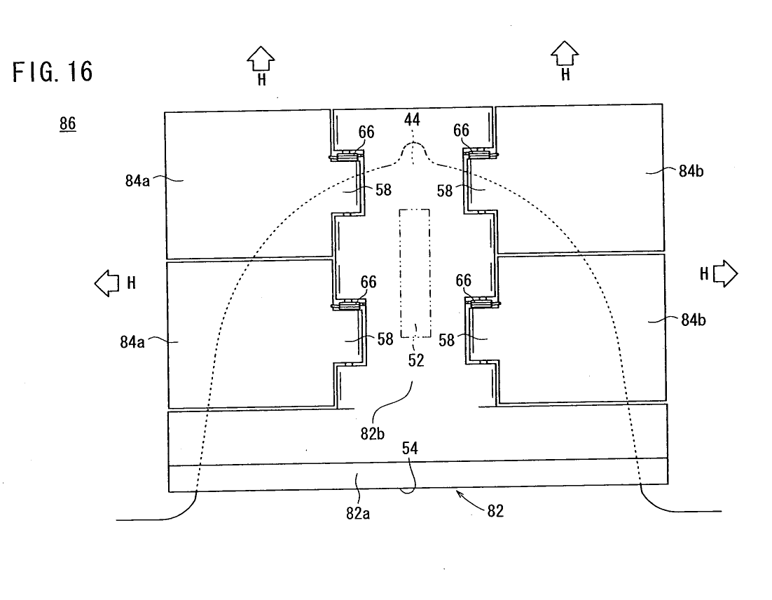

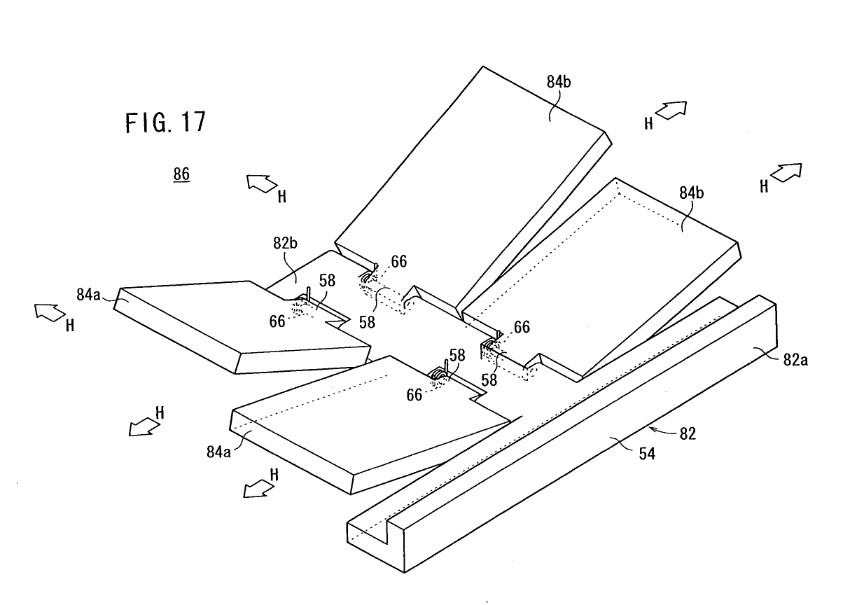

A breast compression plate according to a fifth embodiment of the present invention shall be described below with reference to FIGS. 16 and 17.

The breast compression plate 86 according to the fifth embodiment differs from the breast compression plate 80 according to the fourth embodiment in that helical torsion springs 66 (resilient members) are disposed respectively near the hinges 58, and further, the breast compression plate 86 is free of the distal end bar 82c and the lock mechanisms 62.

When the breast 44 that is held in position by the hand of the operator is compressed by the breast compression plate 86, the proximal portion of the breast 44 and the transversely central portion of the breast 44, which ranges from the proximal to distal ends thereof, are firmly secured in position by the fixed base 82. At this time, the movable flaps 84a, 84b are angularly displaced away from the image capturing base 36 against the bias of the helical torsion springs 66, while lightly compressing the hand of the operator.

Therefore, the hand of the operator and the breast 44 are not excessively compressed. The operator can easily remove his or her hand in any one of the directions indicated by the arrows H shown in FIGS. 16 and 17. After the hand of the operator is removed, the movable flaps 84a, 84b uniformly compress the breast 44 under the bias of the helical torsion springs 66. The breast 44 is thus firmly secured in a desired position between the breast compression plate 86 and the image capturing base 36.

In the mammographic system 12 incorporating the breast compression plate 86 according to the fifth embodiment, the operator's hand is prevented from becoming pinched excessively between the breast compression plate 86 and the image capturing base 36, and hence the breast 44 is prevented from being excessively compressed by the operator's hand. The operator can remove his or her hand from the breast compression plate 86 in a short period of time. The burden on the operator who works with the breast compression plate 86 is greatly reduced, and any pain felt by the operator when his or her hand is placed between the breast compression plate 86 and the image capturing base 36 is also significantly reduced. The burden imposed on the examinee 32, and pain felt when the examinee's breast 44 is compressed, also are greatly reduced. The breast compression plate 86 according to the fifth embodiment may include the lock mechanisms 62. If the breast compression plate 86 includes the lock mechanisms 62, then the fixed base 82 may have a distal end bar 82c, as with the breast compression plate 80 according to the fourth embodiment.

As with the breast compression plate 64, the movable flaps 84a, 84b may be angularly displaced, from their position horizontally aligned with the main compression foot 82b, toward the image capturing base 36.

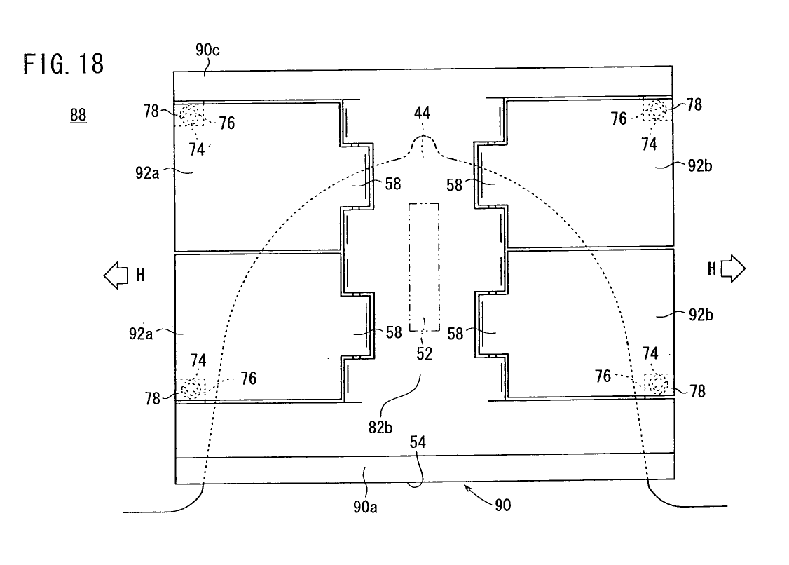

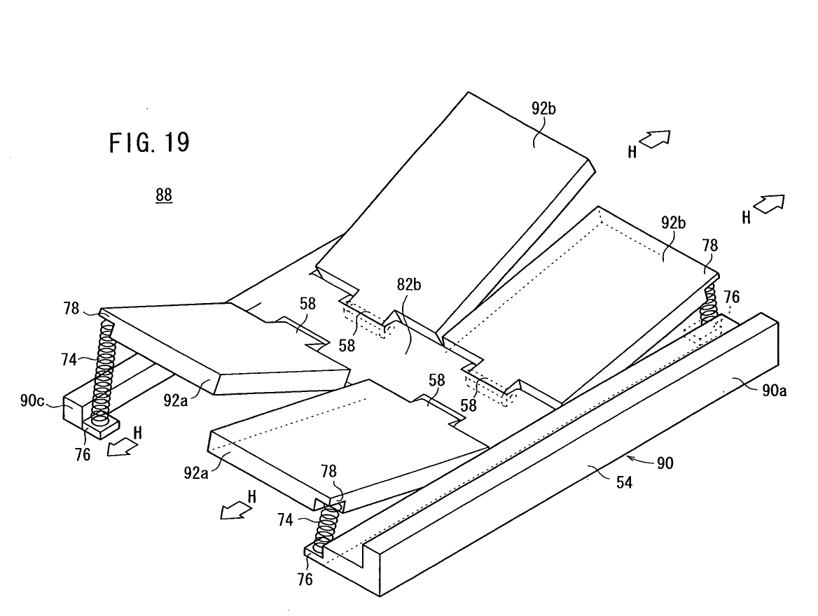

A breast compression plate according to a sixth embodiment of the present invention shall be described below with reference to FIGS. 18 and 19.

The breast compression plate 88 according to the sixth embodiment differs from the breast compression plate 80 according to the fourth embodiment in that it has a fixed base 90 and movable flaps 92a, 92b, in place of the fixed base 82 and the movable flaps 84a, 84b, and further is free of the lock mechanisms 62.

The fixed base 90 also has a base bar 90a and a distal end bar 90c, in place of the base bar 82a and the distal end bar 82c of the fixed base 82. The base bar 90a has two mounts 76 on an end thereof facing the movable flaps 92a, 92b, and also the distal end bar 90c has two mounts 76 on an end thereof facing the movable flaps 92a, 92b. The movable flaps 92a, 92b are different from the movable flaps 84a, 84b in that they include mounts 78 on which helical springs 74 are mounted.

The helical springs 74 act for normally biasing the movable flaps 92a, 92b to turn toward the image capturing base 36. Consequently, when the breast 44 that is held by the hand of the operator is compressed by the breast compression plate 88, the proximal portion of the breast 44 and the transversely central portion of the breast 44, which ranges from the proximal to distal ends thereof, are firmly secured in position by the fixed base 90. At this time, the movable flaps 92a, 92b are angularly displaced away from the image capturing base 36 against the bias of the helical springs 74, while lightly compressing the hand of the operator.

Therefore, the hand of the operator and the breast 44 are not excessively compressed. The operator can easily remove his or her hand in any one of the directions indicated by the arrows H shown in FIGS. 18 and 19. After the hand of the operator is removed, the movable flaps 92a, 92b uniformly compress the breast 44 under the bias of the helical springs 74. The breast 44 is thus firmly secured in a desired position between the breast compression plate 88 and the image capturing base 36.

In the mammographic system 12 incorporating the breast compression plate 88 according to the sixth embodiment, the operator's hand is prevented from becoming pinched excessively between the breast compression plate 88 and the image capturing base 36, and hence the breast 44 is prevented from being excessively compressed by the operator's hand. The operator can remove his or her hand from the breast compression plate 88 in a short period of time. The burden on the operator who works with the breast compression plate 88 is greatly reduced, and any pain felt by the operator when his or her hand is placed between the breast compression plate 88 and the image capturing base 36 is also significantly reduced. The burden imposed on the examinee 32, and pain felt when the examinee's breast 44 is compressed, also are greatly reduced. The breast compression plate 88 according to the sixth embodiment may include the lock mechanisms 62.

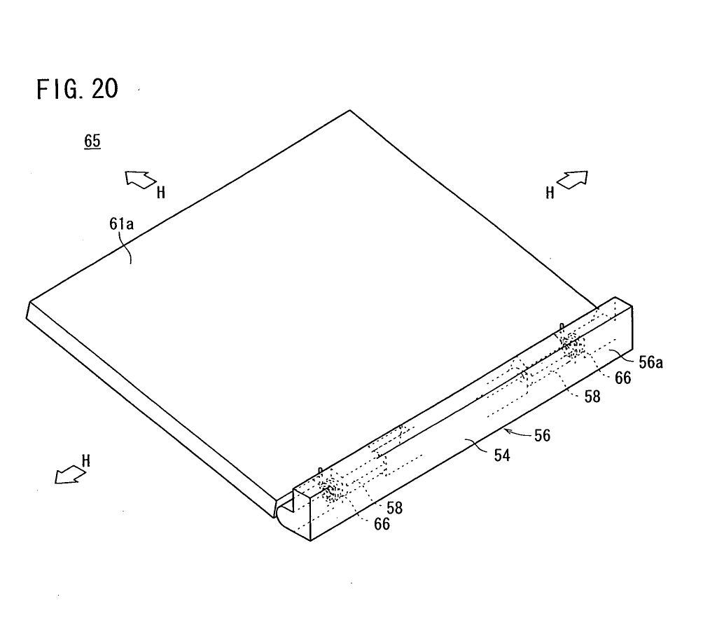

In the above embodiments, the fixed bases 56, 70, 82, 90 and the movable flaps 60a, 60b, 72a, 72b, 84a, 84b, 92a, 92b may be of any shapes other than the shapes shown in the embodiments, insofar as such shapes allow the operator to remove his or her hand easily. For example, a modified breast compression plate 65, as shown in FIG. 20, does not have a main compression foot, but a single movable flap 61a movably connected to a fixed base 56. Although unlike the main compression foot, the single movable flap 61a fails to selectively secure the transversely central portion of the breast 44, which ranges from the proximal to distal ends thereof, the modified breast compression plate 65 is lower in cost and simpler in structure. Therefore, the modified breast compression plate 65 can be used effectively under certain conditions.

The lock mechanisms 62 may include structures other than the ridges 62a and the recesses 62b, insofar as such structures can serve to lock and unlock the movable flaps 60a, 60b, 72a, 72b, 84a, 84b, 92a, 92b.

The breast compression plates 64, 86 include the movable flaps 60a, 60b, 84, 60b, which can be angularly displaced downwardly from their position horizontally aligned with the main compression feet 56b, 86b toward the image capturing base 36. The movable flaps of the breast compression plates 38, 65, 68, 80, 88 according to the other embodiments may also be of the same structure. In such a case, the main compression foot 56b of the breast compression plate 38, for example, may be of a slightly larger thickness, and may have sets of two or three vertically spaced ridges 62a. The main compression foot 70b of the breast compression plate 68, for example, may include mounts 76, which are spaced or offset a greater distance from the upper surface of the main compression foot 70b toward the image capturing base 36.

The parts making up the breast compression plates 38, 64, 65, 68, 80, 86, 88 preferably should be made of a radiation-permeable material.

A cassette housing a stimulable phosphor sheet or an X-ray film therein may be employed instead of the solid-state detector 46. A stimulable phosphor sheet includes a stimulable phosphor layer which, when exposed to applied radiation, stores a portion of the energy of the radiation. Then, when subsequently exposed to applied stimulating rays, such as a laser beam or visible light, the stimulable phosphor layer emits stimulated light in proportion to the stored energy of the radiation.

Although certain preferred embodiments of the present invention have been shown and described in detail, it should be understood that various changes and modifications may be made to the embodiments without departing from the scope of the invention set forth in the appended claims.

What is claimed is:

1. A mammographic system for capturing a radiation image of a breast of an examinee, comprising:
an image capturing base having a radiation image information acquisition unit for acquiring information of the radiation image; and
a breast compression plate for compressing the breast against said image capturing base;
said breast compression plate having a fixed base for compressing and securing the breast, said fixed base including a surface to be held against a chest wall of the examinee, and a movable flap connected to said fixed base and which is displaceable in order to release the breast.

2. A mammographic system according to claim 1, wherein said breast compression plate has a lock mechanism for locking said movable flap against displacement.

3. A mammographic system according to claim 1, wherein said fixed base comprises:
a base bar having said surface to be held against a chest wall of the examinee; and
a main compression foot extending from said base bar in a direction away from said chest wall.

4. A mammographic system according to claim 3, wherein said movable flap is angularly movable by a pivot mechanism mounted on one of said base bar and said main compression foot.

5. A breast compression plate for compressing a breast of an examinee against an image capturing base of a mammographic system for capturing a radiation image of the breast, comprising:
a fixed base for compressing and securing the breast, said fixed base including a surface to be held against a chest wall of the examinee; and
a movable flap connected to said fixed base and which is displaceable in order to release the breast.

6. A breast compression plate according to claim 5, further comprising:
a lock mechanism for locking said movable flap against displacement.

7. A breast compression plate according to claim 6, wherein said lock mechanism locks said movable flap to said fixed base.

8. A breast compression plate according to claim 5, wherein said fixed base comprises:
a base bar having said surface to be held against a chest wall of the examinee; and
a main compression foot extending from said base bar in a direction away from said chest wall.

9. A breast compression plate according to claim 8, wherein said movable flap is angularly movable by a pivot mechanism mounted on one of said base bar and said main compression foot.

10. A breast compression plate according to claim 9, wherein said pivot mechanism has a resilient member for biasing said movable flap in a direction toward said fixed base.

11. A breast compression plate according to claim 9, further comprising:
a lock mechanism for locking said movable flap against angular movement;
said lock mechanism comprising a first engaging member mounted on said movable flap and a second engaging member mounted on said base bar or said main compression foot, said first engaging member and said second engaging member being engageable with each other in order to lock said movable flap against angular movement.

12. A breast compression plate according to claim 9, further comprising:
an extensible and contractible member for biasing said movable flap in a direction toward said fixed base, said extensible and contractible member having an end mounted on said movable flap and an opposite end mounted on one of said base bar and said main compression foot;
said movable flap being biased toward said base bar or said main compression foot by said extensible and contractible member.

13. A breast compression plate according to claim 12, wherein said extensible and contractible member is mounted on a first mount disposed on said movable flap and a second mount disposed on one of said base bar and said main compression foot;
said first mount and said second mount being disposed in confronting relation to each other.
